(19) United States
(12) Patent Application Publication
(10) Pub. No.: US 2026/0365609 A1
(43) Pub. Date: Nov. 19, 2026

(54) PROCESS OF PRODUCING HIGHLY PURE ALKYL ACETATES

(71) Applicant: Sepehr KARIMI ALAGHEHBAND, Isfahan (IR)

(72) Inventor: Sepehr KARIMI ALAGHEHBAND, Isfahan (IR)

(21) Appl. No.: 19/126,089

(22) PCT Filed: Nov. 2, 2022
(86) PCT No.: PCT/IB2022/060560

§ 371 Date: Apr. 30, 2025

(51) Int. Cl. C07C 67/08; C07C 67/54

(52) U.S. Cl. CPC C07C 67/08; C07C 67/54

(57) ABSTRACT

A process of producing high-purity C1-C2 alkyl acetates comprising introducing a feed stream comprising acetic acid, a C1-C2 aliphatic alcohol, and an acid catalyst to a reactive extraction column, contacting the feed stream with an extraction solvent in the first reactive extraction column during an esterification reaction, where the feed stream reacts in the presence of the extraction solvent to produce a reaction product comprising a C1-C2 alkyl acetate and water, removing the C1-C2 alkyl acetate during the reaction from the first reactive extraction column as an overhead stream through solvent extraction. The process further comprises second and third reactive extraction columns for producing and simultaneously extracting as much as possible C1-C2 alkyl acetate through an equilibrium esterification reaction. High-purity C1-C2 alkyl acetates are separated from the extraction solvent using atmospheric distillation.

### TECHNICAL FIELD

The present disclosure is generally related to a process of producing alkyl acetates, particularly to a process of producing highly pure alkyl acetates using the reactive extraction system, and more particularly to a process of producing highly pure methyl and ethyl acetate.

### BACKGROUND

One of the most common commercial production processes of alkyl acetates especially methyl and ethyl acetate is the direct esterification of methanol/ethanol with acetic acid with an acid catalyst. However, this process has encountered difficulty in driving the esterification reactions to completion, thereby resulting in acetate products contaminated with unreacted alcohols, which can result in a relatively expensive separation system to purify the product. Accordingly, the improvements in the production of acetates have dealt mainly with finding alternative reaction processes to produce pure acetates.

Other improvements for alkyl acetates production such as reactive distillation or extractive distillation systems need several complementary purification steps that are expensive and highly energy-consuming. Consequently, there is a need to develop a cost-effective and highly robust industrial process for the production of highly pure alkyl acetates using low-cost equipment with safe reaction conditions without any need for high temperature, and high pressure. Moreover, there is a need for an efficient process for producing highly pure alkyl acetates with high yield and the ability to recycle the extraction solvent.

### SUMMARY

This summary is intended to provide an overview of the subject matter of the present disclosure, and is not intended to identify essential elements or key elements of the subject matter, nor is it intended to be used to determine the scope of the claimed implementations. Its sole purpose is to present some concepts of one or more aspects in a simplified form as a prelude to the more detailed description that is presented later. The proper scope of the present disclosure may be ascertained from the claims set forth below in view of the detailed description below and the drawings.

In one general aspect, the present disclosure describes an exemplary process of producing highly pure alkyl acetates. In one exemplary embodiment, the process may include producing a first mixture by mixing acetic acid, a C1-C2 aliphatic alcohol, and an acid catalyst in first mixer, and then atomizing the first mixture using a first atomizer. In one exemplary embodiment, producing C1-C2 alkyl acetate with at least 95% purity may include producing a first reaction product comprising a C1-C2 alkyl acetate and water in the first reactive extraction column, wherein the conversion of the C1-C2 aliphatic alcohol in the atomized first mixture to the C1-C2 alkyl acetate is at maximum 70%. In one exemplary embodiment, removing high-purity C1-C2 alkyl acetates produced in the first reactive extraction column may include removing an overhead stream from the first reactive extraction column by contacting the first reaction product with a first extraction solvent, wherein the overhead stream comprises a first organic phase comprising 10% wt. C1-C2 alkyl acetate produced in the first reaction product that is dissolved in the first extraction solvent. In one exemplary embodiment, producing C1-C2 alkyl acetate with at least 95% purity may include separating the first organic phase into C1-C2 alkyl acetate with at least 95% purity and the first extraction solvent using the first distillation unit, wherein separating the first organic phase into C1-C2 alkyl acetate with at least 95% purity and the first extraction solvent using the first distillation unit comprises regenerating the first extraction solvent. In one exemplary embodiment, the regenerated first extraction solvent is recycled back to the first reactive extraction column.

In one exemplary embodiment, producing C1-C2 alkyl acetate with at least 95% purity may include transferring a first residual compound in the first reactive extraction column to a second atomizer, wherein the first residual compound comprising unreacted acetic acid, the C1-C2 aliphatic alcohol, the acid catalyst, water, and C1-C2 alkyl acetate produced in the first reaction product are atomized. In one exemplary embodiment, producing C1-C2 alkyl acetate with at least 95% purity may include producing a second reaction product comprising a C1-C2 alkyl acetate and water in the second reactive extraction column, wherein the conversion of the C1-C2 aliphatic alcohol in the atomized first residual compounds to the C1-C2 alkyl acetate is at maximum 90%. In one exemplary embodiment, removing high-purity C1-C2 alkyl acetates produced in the second reactive extraction column may include removing an overhead stream from the second reactive extraction column by contacting the second reaction product with the second extraction solvent, wherein the overhead stream comprises a second organic phase comprising 10% wt. C1-C2 alkyl acetate produced in the second reaction product that is dissolved in the second extraction solvent. In one exemplary embodiment, producing C1-C2 alkyl acetate with at least 95% purity may include separating the second organic phase into C1-C2 alkyl acetate with at least 95% purity and the second extraction solvent using the second distillation unit, wherein separating the second organic phase into C1-C2 alkyl acetate with at least 95% purity and the second extraction solvent using the second distillation unit comprises regenerating the second extraction solvent. In one exemplary embodiment, the regenerated second extraction solvent is recycled back to the second reactive extraction column.

In one exemplary embodiment, producing C1-C2 alkyl acetate with at least 95% purity may include transferring a second residual compound in the second reactive extraction column to a second mixer, wherein the second residual compounds comprising unreacted acetic acid, the C1-C2 aliphatic alcohol, the acid catalyst, water, and C1-C2 alkyl acetate produced in the second reaction product. In one exemplary embodiment, producing C1-C2 alkyl acetate with at least 95% purity may include producing a second mixture by adding a 10% wt. salt solution to the second mixer, wherein the second residual compound and salt solution are mixed, and then atomizing the second mixture using a third atomizer. In one exemplary embodiment, the salt solution may be prepared using a salt having 10% wt. solubility in water. Salts may include, but are not limited to, calcium chloride, sodium sulfate, sodium acetate, potassium chloride. In one exemplary embodiment, producing C1-C2 alkyl acetate with at least 95% purity may include contacting the atomized second mixture with the third extraction solvent in the third reactive extraction column, wherein the C1-C2 alkyl acetate remaining in the atomized second mixture is extracted by the third extraction solvent. In one exemplary embodiment, producing C1-C2 alkyl acetate with at least 95% purity may include removing an overhead stream from the third reactive extraction column, wherein the overhead stream comprises a third organic phase comprising 10% wt. C1-C2 alkyl acetate remained in the atomized second mixture that is dissolved in the third extraction solvent. In one exemplary embodiment, producing C1-C2 alkyl acetate with at least 95% purity may include separating the third organic phase into C1-C2 alkyl acetate with at least 95% purity and the third extraction solvent using the third distillation unit, wherein separating the third organic phase into C1-C2 alkyl acetate with at least 95% purity and the third extraction solvent using the third distillation unit comprises regenerating the third extraction solvent. In one exemplary embodiment, the regenerated third extraction solvent is recycled back to the third reactive extraction column.

This Summary may introduce a number of concepts in a simplified format; the concepts are further disclosed within the “Detailed Description” section. This Summary is not intended to configure essential/key features of the claimed subject matter, nor is intended to limit the scope of the claimed subject matter.

### BRIEF DESCRIPTION OF THE DRAWINGS

The present disclosure is disclosed more in detail with reference to the drawings in which:

FIG. 1 is a schematic piping and instrumentation diagram (P&ID) of producing high-purity C1-C2 alkyl acetate, consistent with one or more exemplary embodiments of the present disclosure.

FIG. 2 shows a flowchart of an exemplary process for producing high-purity C1-C2 alkyl acetate in first reactive extraction column, consistent with one or more exemplary embodiments of the present disclosure.

FIG. 3 shows a flowchart of an exemplary process for producing high-purity C1-C2 alkyl acetate in second reactive extraction column, consistent with one or more exemplary embodiments of the present disclosure.

FIG. 4 shows a flowchart of an exemplary process for producing high-purity C1-C2 alkyl acetate in third reactive extraction column, consistent with one or more exemplary embodiments of the present disclosure.

It is understood that the following description and references to the figures concern exemplary embodiments of the present disclosure and shall not be limiting the scope of the claims.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well-known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown but is to be accorded the widest possible scope consistent with the principles and features disclosed herein.

Disclosed herein is an exemplary cost-effective and straightforward reactive extraction system and process for producing highly pure C1-C2 alkyl acetates through an esterification reaction. “Highly pure alkyl acetate” and “high-purity alkyl acetate” may refer to at least 95% alkyl acetate by weight. In an exemplary implementation, the reactive extraction system may be used for producing highly pure C1-C2 alkyl acetates, which may not only decrease the costs of the common methods but may also obviate the need for high temperature and high pressure, which may be considered as industrial obstacles. In one or more exemplary implementations, an exemplary C1-C2 alkyl acetate may be produced through an esterification reaction of an exemplary C1-C2 aliphatic alcohol with acetic acid in the presence of an exemplary acid catalyst at temperatures between 20 °C and 35 °C. An exemplary process may further comprise the separation of an exemplary produced C1-C2 alkyl acetate from one or more exemplary reactants and byproducts through dissolving C1-C2 alkyl acetate in an exemplary extraction solvent. An exemplary process may be considered as an efficient process for producing C1-C2 alkyl acetates with high-purity by distilling the solution containing the produced C1-C2 alkyl acetate and extraction solvent. An exemplary process may allow for a one-step C1-C2 alkyl acetate production process, which may be advantageous relative to other processes that require further steps to purify the C1-C2 alkyl acetates. Each of these advantages may be provided in a process that may also be less expensive than alternative processes by C1-C2 alkyl acetates production using the esterification reactions. Furthermore, as advantages of the integration of the reaction and extraction, esterification equilibrium limitations may be overcome, and the reaction may be carried out at temperatures between 20 °C and 35 °C, under atmospheric pressure. In one or more exemplary processes, the extraction solvent may be recycled into an exemplary reaction column. According to one or more exemplary embodiments, C1-C2 alkyl acetates may be produced using an exemplary continuous, semi-continuous, or batch processes. Using one or more exemplary embodiments of the processes, high-purity methyl acetate and/or ethyl acetate may be produced.

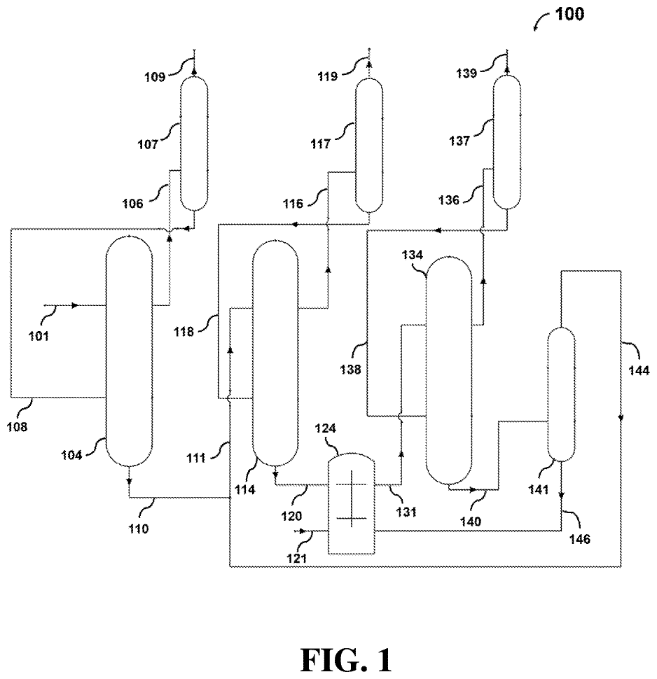

FIG. 1 is a schematic piping and instrumentation diagram (P&ID) 100 of producing high-purity C1-C2 alkyl acetates, consistent with one or more exemplary embodiments of the present disclosure. Exemplary P&ID 100 may include reactive extraction column 104, reactive extraction column 114, and reactive extraction column 134 for producing high-purity C1-C2 alkyl acetates through an esterification reaction. “Reactive extraction column” may refer to a reaction column in which both reaction and extraction are performed. An exemplary reactive extraction column may have trays, packing, or some other type of complex internal structure. Examples of reactive extraction columns may include scrubbers, strippers, absorbers, adsorbers, packed columns, and columns having valve, sieve, or other types of trays. Exemplary reactive extraction columns may employ weirs, downspouts, internal baffles, temperature control elements, and/or pressure control elements. In one or more exemplary embodiments, C1-C2 alkyl acetates may be produced via an esterification reaction of the acetic acid with an exemplary C1-C2 aliphatic alcohol in the presence of an acid catalyst in one or more reactive extraction columns. “Esterification reaction” may refer to a process of producing an ester (RCOOR) and water by combining a carboxylic acid with an alcohol.

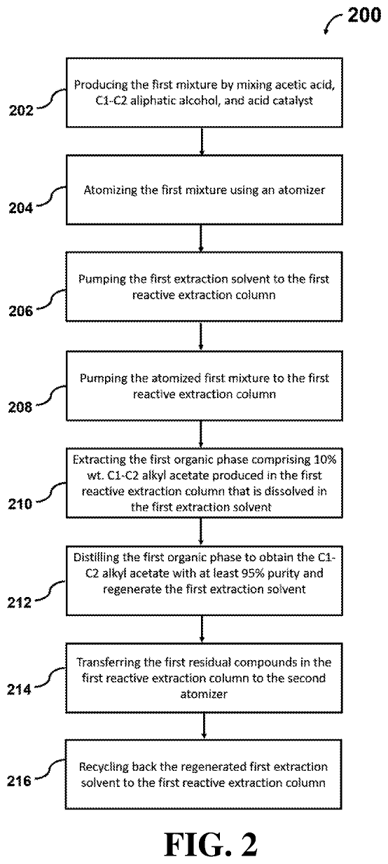

In further detail with regards to an exemplary system and process of producing high-purity esters in first reactive extraction column 104, FIG. 2 shows a flowchart of exemplary process 200 for producing high-purity C1-C2 alkyl acetate in first reactive extraction column 104, consistent with one or more exemplary embodiments of the present disclosure. An exemplary process 200 may include producing the first mixture by mixing acetic acid, a C1-C2aliphatic alcohol, and an acid catalyst (step 202), atomizing the first mixture using an atomizer (step 204), pumping the first extraction solvent to the first reactive extraction column (step 206), pumping the atomized first mixture to the first reactive extraction column (step 208), extracting the first organic phase comprising 10% wt. C1-C2 alkyl acetate produced in the first reactive extraction column that is dissolved in the first extraction solvent (step 210), distilling the first organic phase to obtain the C1-C2 alkyl acetate with at least 95% purity and regenerate the first extraction solvent (step 212), transferring the first residual compounds in the first reactive extraction column to the second atomizer (step 214), and recycling back the regenerated first extraction solvent to the first reactive extraction column (step 216).

Referring to FIG. 1 and FIG. 2, in one or more exemplary embodiments, step 202 may include producing the first mixture by mixing acetic acid, a C1-C2 aliphatic alcohol, and an acid catalyst. In an exemplary embodiment, forming the first mixture may include mixing the reactants in a first mixer at controlled temperatures in the range of 20 °C and 35 °C, results in preparing the first mixture. In one or more exemplary embodiments, mixing a C1-C2 aliphatic alcohol with acetic acid may include mixing a C1-C2 aliphatic alcohol with acetic acid with a molar ratio between 1.1 to 1.3 in the presence of at least 1% wt. acid catalyst based on acetic acid. Acid catalyst may include, but is not limited to, sulfuric acid. In one exemplary embodiment, mixing a C1-C2 aliphatic alcohol with acetic acid may include mixing a C1-C2 aliphatic alcohol with acetic acid with a molar ratio 1.2 in the presence of at least 1% wt. acid catalyst based on acetic acid.

In one or more exemplary embodiments, step 204 may include atomizing the first mixture using an atomizer. In one exemplary embodiment, atomizing the first mixture using an atomizer may include transferring the first mixture to a first atomizer for increasing the contact area of the reactants mixture droplet via decreasing their size via atomizing the mixture. “Atomizer” may refer to a device for converting the substances into very fine particles or droplets.

In one or more exemplary embodiments, step 206 may include pumping the first extraction solvent to the first reactive extraction column. In one exemplary embodiment, pumping the first extraction solvent to the first reactive extraction column may include pumping the first extraction solvent into first reactive extraction column 104. The esterification reaction for C1-C2 alkyl acetate production may initiate and carry on in first reactive extraction column 104 in the presence of a sufficient amount of the first extraction solvent which is adjusted regarding the level of the compounds in first reactive extraction column 104. Thereby, the need for much first extraction solvent may be balanced by a dosing pump via pumping the first extraction solvent into first reactive extraction column 104. The first extraction solvent may have a boiling point between temperatures of 140 °C and 220 °C and a density between 0.78 g/cm³ and 0.8 g/cm³. The first extraction solvent may include, but is not limited to, a combination of 5% wt. aromatic compound, and 95% wt. of a mixture of naphthenic and paraffinic compounds, wherein the mixture of the naphthenic compound and the paraffinic compound has a weight ratio (the naphthenic compound: the paraffinic compound) ranging from 20:80 to 80:20.

In one or more exemplary embodiments, step 208 may include pumping the atomized first mixture to the first reactive extraction column. In one exemplary embodiment, pumping the atomized first mixture to the first reactive extraction column may include feeding atomized first mixture feed 101 to first reactive extraction column 104 from the top of first reactive extraction column 104 as an overhead stream. In one exemplary embodiment, the first mixture may be transferred to first reactive extraction column 104 using a pump.

In one or more exemplary embodiments, an exemplary C1-C2 alkyl acetate may be produced in first reactive extraction column 104 through an esterification reaction at temperatures between 20 °C and 35 °C, and atmospheric pressure, results in producing the first reaction product. In one exemplary embodiment, the first reaction product in the first reactive extraction column 104 comprises a C1-C2 alkyl acetate and water. An exemplary produced C1-C2 alkyl acetate may be incompatible with the residual reactants, containing acetic acid and aliphatic alcohol, and water as the byproduct of the esterification reaction. On the contrary, the first extraction solvent may be designed to be as much as compatible with the produced C1-C2 alkyl acetate. In one or more exemplary embodiments, step 210 may include extracting the first organic phase comprising 10% wt. C1-C2 alkyl acetate produced in the first reactive extraction column that is dissolved in the first extraction solvent. Extracting the first organic phase comprising 10% wt. C1-C2 alkyl acetate produced in the first reactive extraction column that is dissolved in the first extraction solvent may include extracting an exemplary first organic phase comprising produced C1-C2 alkyl acetate dissolved in the first extraction solvent, in which the produced C1-C2 alkyl acetate may be separated from the other compounds in first reactive extraction column 104 via dissolving in the first extraction solvent and leaving the reaction environment from the top of first reactive extraction column 104 as an overhead stream 106. In one exemplary embodiment, the conversion of the C1-C2 aliphatic alcohol in the atomized first mixture to the C1-C2 alkyl acetate in the first reactive extraction column 104 is at maximum 70%. In one or more exemplary embodiments, overhead product stream 106 may comprise 10% wt. C1-C2 alkyl acetate produced in column 104 in the first reaction product dissolved in the first extraction solvent.

In one or more exemplary embodiments, step 212 may include distilling the first organic phase to obtain the C1-C2 alkyl acetate with at least 95% purity and regenerate the first extraction solvent. In one or more exemplary embodiments, distilling the first organic phase to obtain the C1-C2 alkyl acetate with at least 95% purity and regenerate the first extraction solvent may include transferring the first organic phase as an overhead stream 106 into an exemplary first atmospheric distillation unit 107 as a bottoms stream, wherein the high-purity C1-C2 alkyl acetate and first extraction solvent may be separated. An exemplary high-purity C1-C2 alkyl acetate 109 may be collected through an overhead stream from the top of first distillation unit 107. In an embodiment, overhead product stream 109 may comprise greater than about 90%, greater than about 95%, greater than about 96%, greater than about 97%, greater than about 98%, greater than about 99%, or greater than about 99.5% C1-C2 alkyl acetate by weight.

In one or more exemplary embodiments, step 214 may include transferring the first residual compounds in the first reactive extraction column to the second atomizer. In one exemplary embodiment, transferring the first residual compounds in the first reactive extraction column to the second atomizer may include evacuating and transferring an exemplary first remaining bottoms products or first residual products of first reactive extraction column 104 from first reactive extraction column 104 as a bottoms stream 110 into the next system and process of the producing high-purity C1-C2 alkyl acetate. An exemplary first remaining bottoms products or first residual products of first reactive extraction column 104 may be comprised the C1-C2 alkyl acetate produced in the column 104, along with unreacted aliphatic alcohol, acetic acid, acid catalyst, and potentially any side products produced by the esterification reaction such as water. In one or more exemplary embodiments, the conversion of the esterification reaction of the C1-C2 alkyl acetate production in the first reactive extraction column 104 may be about 65% to 70%. It means that a portion of the reactants may remain unreacted, transferred to another reactive extraction system for producing high-purity C1-C2 alkyl acetate.

In one or more exemplary embodiments, step 216 may include recycling back the regenerated first extraction solvent to the first reactive extraction column. In one exemplary embodiment, separating the first organic phase into C1-C2 alkyl acetate with at least 95% purity and the first extraction solvent using the first distillation unit comprises regenerating the first extraction solvent. In one exemplary embodiment, recycling back the regenerated first extraction solvent to the first reactive extraction column may include recycling back the first extraction solvent, which is regenerated in first distillation unit 107 (step 212), into first reactive extraction column 104 as a bottoms stream 108.

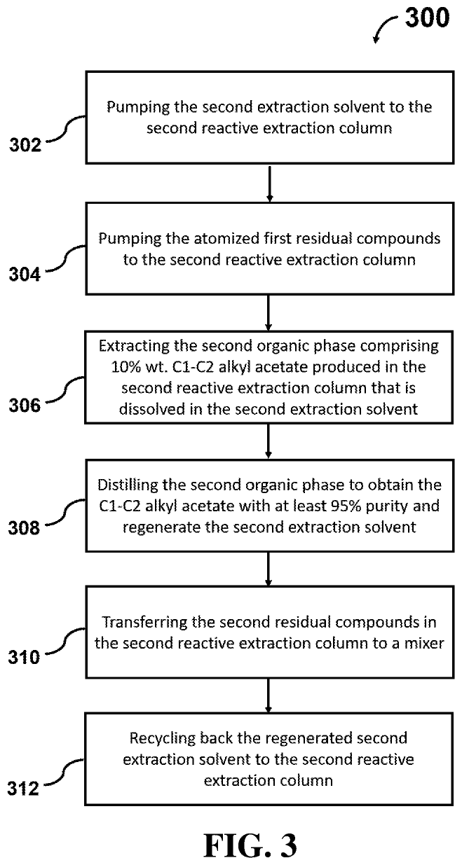

In further detail with respect to an exemplary system and process of producing high-purity C1-C2 alkyl acetate in second reactive extraction column 114, FIG. 3 shows a flowchart of an exemplary process 300 for producing high-purity C1-C2 alkyl acetate in second reactive extraction column 114, consistent with one or more exemplary embodiments of the present disclosure. An exemplary process 300 may include pumping the second extraction solvent to the second reactive extraction column (step 302), pumping the atomized first residual compounds to the second reactive extraction column (step 304), extracting the second organic phase comprising 10% wt. C1-C2 alkyl acetate produced in the second reactive extraction column that is dissolved in the second extraction solvent (step 306), distilling the second organic phase to obtain the C1-C2 alkyl acetate with at least 95% purity and regenerate the second extraction solvent (step 308), transferring the second residual compounds in the second reactive extraction column to a second mixer (step 310), and recycling back the regenerated second extraction solvent to the second reactive extraction column (step 312).

Referring to FIG. 1 and FIG. 3, exemplary process 300 may include sending an exemplary first residual compounds, as bottoms stream 110, from an exemplary first reactive extraction column 104 (see FIG. 1) to an exemplary second reactive extraction column 114 through feed stream 111 that may comprise first residual compounds 110 and co-feed stream 144. In an exemplary embodiment, first residual compound 110 may comprise unextracted C1-C2 alkyl acetate produced in first reactive extraction column 104, along with unreacted aliphatic alcohol, acetic acid, acid catalyst, and water. In one or more exemplary embodiments, feed stream 111 may be passed through a second atomizer before transferring to an exemplary second reactive extraction column 114. In one or more exemplary embodiments, step 302 may include pumping an exemplary second extraction solvent into second reactive extraction column 114. An exemplary second extraction solvent may have a boiling point between temperatures of 150 °C and 220 °C and a density between 0.72 g/cm³ and 0.74 g/cm³. An exemplary second extraction solvent may include, but is not limited to, a combination of 20% wt. aromatic compound having boiling points between 180 °C and 190 °C, and 80% wt. of a mixture of naphthenic and paraffinic compounds, wherein the mixture of the naphthenic compound and the paraffinic compound has a weight ratio (the naphthenic compound: the paraffinic compound) ranging from 20:80 to 80:20. The need for much second extraction solvent may be balanced by a dosing pump via pumping the second extraction solvent into second reactive extraction column 114 as a co-feed 118, as described below.

In one or more exemplary embodiments, step 304 may include pumping the atomized first residual compounds to the second reactive extraction column. In one exemplary embodiment, pumping the atomized first residual compounds to second reactive extraction column 114 as an overhead stream 111 from the top of second reactive extraction column 114 may include transferring the first residual compounds to a second atomizer, wherein the compounds may convert to very fine droplets with more surface area increasing the efficiency of the C1-C2 alkyl acetate extraction due to increasing the contact area of the reactants mixture droplets, results in moving the esterification reaction forward. In one exemplary embodiment, pumping the atomized first residual compounds to second reactive extraction column 114 as an overhead stream 111 from the top of second reactive extraction column 114 may further include transferring the atomized first residual compounds to second reactive extraction column 114 using a pump. The operating temperature of feed stream 111 to column 114 may be controlled between 25 °C and 40 °C.

Referring again to FIG. 1 and FIG. 3, in one or more exemplary embodiments, the esterification reaction of the residual unreacted reactants transferred to second reactive extraction column 114 through feed stream 111 may progress for assessing the efficiency about 70%, greater than about 80%, greater than about 90%. In one or more exemplary embodiments, step 306 may include extracting the second organic phase comprising 10% wt. C1-C2 alkyl acetate produced in the second reactive extraction column that is dissolved in the second extraction solvent. In one exemplary embodiment, extracting the second organic phase comprising 10% wt. C1-C2 alkyl acetate produced in the second reactive extraction column that is dissolved in the second extraction solvent may include producing a second reaction product comprising C1-C2 alkyl acetate and water in the second reactive extraction column, wherein the conversion of the C1-C2 aliphatic alcohol in the atomized first residual compounds to the C1-C2 alkyl acetate is at maximum 90%. In one exemplary embodiment, step 306 may further include extracting an exemplary second organic phase comprising 10% wt. C1-C2 alkyl acetate produced in the second reactive extraction column that is dissolved in the second extraction solvent, in which the produced C1-C2 alkyl acetate may be separated from the other compounds in second reactive extraction column 114 via dissolving in the second extraction solvent and leaving the reaction environment from the top of second reactive extraction column 114 as an overhead stream 116. Indeed, relatively high conversion of the esterification reaction in column 114 may be achieved via overcoming the reaction equilibrium by removing the produced C1-C2 alkyl acetates through the extraction. In one or more exemplary embodiments, overhead product stream 116 may comprise 10% wt. C1-C2 alkyl acetate produced in the second reaction product that is dissolved in the second extraction solvent.

In one or more exemplary embodiments, step 308 may include distilling the second organic phase to obtain the C1-C2 alkyl acetate with at least 95% purity and regenerate the second extraction solvent. In one exemplary embodiment, distilling the second organic phase to obtain the C1-C2 alkyl acetate with at least 95% purity and regenerate the second extraction solvent may include transferring the second organic phase exited as an overhead stream 116 from column 114 to an exemplary second atmospheric distillation unit 117 as a bottoms stream, wherein the high-purity C1-C2 alkyl acetate and second extraction solvent may be separated. An exemplary high-purity C1-C2 alkyl acetate 119 may be collected through an overhead stream from the top of second distillation unit 117. In an embodiment, overhead product stream 119 may comprise greater than about 90%, greater than about 95%, greater than about 96%, greater than about 97%, greater than about 98%, greater than about 99%, or greater than about 99.5% C1-C2 alkyl acetate by weight. In one exemplary embodiment, separating the second organic phase into C1-C2 alkyl acetate with at least 95% purity and the second extraction solvent using the second distillation unit may include regenerating the second extraction solvent.

In one or more exemplary embodiments, step 310 may include transferring the second residual compounds in the second reactive extraction column to a second mixer. In one or more exemplary embodiments, transferring the second residual compounds in the second reactive extraction column to a second mixer may include evacuating and transferring an exemplary second remaining bottoms products or second residual products of second reactive extraction column 114 from second reactive extraction column 114 as a bottoms stream 120 into second mixer 124 for modifying before entering the next system and process of the producing high-purity C1-C2 alkyl acetate. “Modification” may refer to some treatment steps which are described below.

In one or more exemplary embodiments, step 312 may include recycling back the regenerated second extraction solvent to the second reactive extraction column. In one exemplary embodiment, the second extraction solvent which is regenerated in second distillation unit 117 (step 308) is recycled back into second reactive extraction column 114 as bottoms stream 118. In an exemplary embodiment, the regenerated second solvent extraction may comprise about 5% C1-C2 alkyl acetate in a continuous system and process of producing high-purity C1-C2 alkyl acetate.

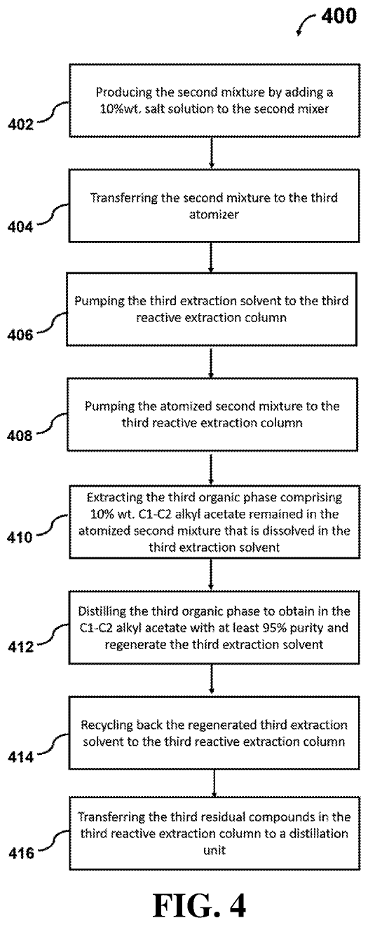

In further detail with respect to an exemplary system and process of producing high-purity C1-C2 alkyl acetates in the third reactive extraction column 134, FIG. 4 shows a flowchart of an exemplary process 400 for producing high-purity C1-C2 alkyl acetates in third reactive extraction column 134, consistent with one or more exemplary embodiments of the present disclosure. An exemplary process 400 may include producing the second mixture by adding a 10% wt. salt solution to the second mixer (step 402), transferring the second mixture to the third atomizer (step 404), pumping the third extraction solvent to the third reactive extraction column (step 406), pumping the atomized second mixture to the third reactive extraction column (step 408), extracting the third organic phase comprising 10% wt. C1-C2 alkyl acetate remained in the atomized second mixture that is dissolved in the third extraction solvent (step 410), distilling the third organic phase to obtain in the C1-C2 alkyl acetate with at least 95% purity and regenerate the third extraction solvent (step 412), recycling back the regenerated third extraction solvent to the third reactive extraction column (step 414), and transferring the third residual compounds in the third reactive extraction column to a distillation unit.

Referring to FIG. 1 and FIG. 4, FIG. 4 shows a flowchart of an exemplary process 400 where second residual compounds as a bottoms stream 120 from second reactive extraction column 114 illustrated in FIG. 1 is sent to second mixer 124. In an exemplary embodiment, second residual compound 120 may comprise unextracted C1-C2 alkyl acetates produced in second reactive extraction column 114 in the second reaction product, along with unreacted C1-C2 aliphatic alcohol, acetic acid, acid catalyst, and water. In one or more exemplary embodiments, step 402 may include producing the second mixture by adding a 10% wt. salt solution 121 to second mixer 124, wherein may be mixed with second residual compound 120. In one exemplary embodiment, the salt solution may be prepared using a salt having 10% wt. solubility in water. Salts may include, but are not limited to, calcium chloride, sodium sulfate, sodium acetate, potassium chloride. An exemplary second mixture in second mixer 124 may be diluted using an exemplary residual compound of fourth distillation unit 141 which may be evacuated as a bottoms stream 146 from fourth distillation unit 141 and transferred to second mixer 124 as a co-feed. An exemplary residual compound of fourth distillation unit 141 may be comprised an acid catalyst and water which may be neutralized and used as a diluent for preparing the second mixture.

In one or more exemplary embodiments, step 404 may include transferring the second mixture to a third atomizer. Transferring the second mixture to third atomizer may include atomizing the second mixture as an exemplary feed stream of third reactive extraction column 134, before pumping to the column.

In one or more exemplary embodiments, step 406 may include pumping the third extraction solvent to the third reactive extraction column. Pumping the third extraction solvent to the third reactive extraction column may include transferring and pumping an exemplary third extraction solvent into third reactive extraction column 134. The third extraction solvent may have a boiling point between temperatures of 150 °C and 180 °C and a density between 0.77 g/cm³ and 0.79 g/cm³. The third extraction solvent may include, but is not limited to, a combination of 35% wt. aromatic compound, and 65% wt. of a mixture of naphthenic and paraffinic compounds, wherein the mixture of the naphthenic compound and the paraffinic compound has a weight ratio (the naphthenic compound: the paraffinic compound) ranging from 20:80 to 80:20. The need for much third extraction solvent may be balanced by a dosing pump via pumping the third extraction solvent into third reactive extraction column 134 as a co-feed 138, as described below.

In one or more exemplary embodiments, step 408 may include pumping the atomized second mixture to third reactive extraction column. Pumping the atomized second mixture to the third reactive extraction column may include pumping the atomized second mixture to third reactive extraction column 134 as an overhead stream 131 exited from the top of the second mixture 124.

Again referring to FIG. 1 and FIG. 3, in one or more exemplary embodiments, step 410 may include extracting the third organic phase comprising 10% wt. C1-C2 alkyl acetate remained in the atomized second mixture that is dissolved in the third extraction solvent. Extracting the third organic phase comprising 10% wt. C1-C2 alkyl acetate remained in the atomized second mixture that is dissolved in the third extraction solvent may include extracting the third organic phase comprising 10% wt. C1-C2 alkyl acetate remained in the atomized second mixture that is dissolved in the third extraction solvent, in which the remained C1-C2 alkyl acetates may be separated from the other compounds in third reactive extraction column 134 via dissolving in the third extraction solvent and leaving the reaction environment from the top of third reactive extraction column 134 as an overhead stream 136. In one or more exemplary embodiments, the overhead product stream 136 may comprise 10% wt. C1-C2 alkyl acetate dissolved in the third extraction solvent. In one or more exemplary embodiments, no more esterification reaction may happen in third reactive extraction column 134. Therefore, the third extraction solvent may extract the remained C1-C2 alkyl acetates produced in second reactive extraction column 114 which may be transferred to third reactive extraction column 134 through second residual compound 120. In one or more exemplary embodiments, step 412 may include distilling the third organic phase to obtain in the C1-C2 alkyl acetate with at least 95% purity and regenerate the third extraction solvent. The third organic phase may transfer to an exemplary third atmospheric distillation unit 137 as a bottoms stream 136 from the top of column 134. Distilling the third organic phase to obtain in the C1-C2 alkyl acetate with at least 95% purity and regenerate the third extraction solvent may further include separating the high-purity C1-C2 alkyl acetate and the third extraction solvent through third distillation unit 137, wherein separating the third organic phase into C1-C2 alkyl acetate with at least 95% purity and the third extraction solvent using the third distillation unit comprises regenerating the third extraction solvent. An exemplary high-purity C1-C2 alkyl acetate may be collected through an overhead stream 139 from the top of third distillation unit 137. In an embodiment, the overhead product stream 139 may comprise greater than about 90%, greater than about 95%, greater than about 96%, greater than about 97%, greater than about 98%, greater than about 99%, or greater than about 99.5% C1-C2 alkyl acetate by weight.

In one or more exemplary embodiments, step 414 may include recycling back the third extraction solvent, which is regenerated in third distillation unit 137 (step 412), into the third reactive extraction column 134 as a bottoms stream 138. As described above, co-feed stream 138 comprising regenerating third reaction solvent may be used for adjusting the required level of the solvent in column 134.

In one or more exemplary embodiments, step 416 may include evacuating and transferring an exemplary third remaining bottoms products or third residual compounds from third reactive extraction column 134 as a bottoms stream 140 into fourth atmospheric distillation unit 141 for separating the produced C1-C2 alkyl acetate and alcohol from the other compounds. In one or more exemplary embodiments, the third residual compound may be comprised insignificant amounts of remained C1-C2 alkyl acetate in column 134, along with unreacted aliphatic alcohol, acetic acid, acid catalyst, and potentially water may be added. In one or more exemplary embodiments, the separated products from fourth distillation unit 141 may evacuate as an overhead stream 144 and mix with first residual compounds 110 of column 104 before feeding to second reactive extraction column 114. An exemplary residual compound of fourth distillation unit 141 may be comprised an acid catalyst and water which may be neutralized and used as a diluent for preparing the second mixture. Thereby, the residual compound of fourth distillation unit 141 may be evacuated as a bottoms stream 146 from fourth distillation unit 141 and transferred to second mixer 124 as a co-feed.

To better illustrate the objects, aspects and advantages of the present disclosure, the present disclosure will be further described with reference to specific examples. It will be understood by those skilled in the art that the specific embodiments described herein are merely illustrative of the present disclosure and are not intended to limit the present disclosure.

In the examples, the experimental methods used were all conventional methods unless otherwise specified, and the materials, reagents and the like used were commercially available without otherwise specified.

### Example 1: Production of High-Purity Ethyl Acetate

In this example, ethyl acetate was produced by an exemplary reactive extraction system and process similar to exemplary P&ID 100 as presented in FIG. 1. At first, a first mixture of acetic acid, ethanol, and acid sulfuric was prepared in a 1000 cc beaker. The temperature of the first mixture was controlled at about 30 °C. The preparation involved mixing 300 (g) of acetic acid (99.9% purity), 10% excess weight of ethanol (99.9% purity) about 253 (g), with 3 (g) of acid sulfuric (98% purity), and stirred until well mixed. Then, three 20-inch height and 4-inch diameter glass column were packed with either ceramic saddles, stainless steel pall rings, or trays. In the next step, the first column was filled with the first extraction solvent. The volume of the solvent was 2772 g. It should be mentioned that the volume of the solvent is proportional to separating a 10% wt. ethyl acetate solution. Then, the first mixture was added dropwise to the first column using a dropping funnel over 2 hours. Ethyl acetate is dissolved in the first solvent as soon as it is formed through the esterification reaction. Then, unreacted reactants comprising ethanol, acetic acid, and acid sulfuric with water as a potential byproduct produced by the esterification reaction were sedimented at the bottoms of the first reactive extraction column. In the next step, the residual compounds sedimented at the bottoms of the first reactive extraction column were transferred to a dropping funnel for dropwise adding to the second reactive extraction column which is filled with the second solvent. The esterification reaction was completed in the second column. However, about 80% of the produced ethyl acetate was transferred to the solvent phase. Again, the residual compounds were sedimented at the bottoms of the second column which may be evacuated for the next step. Thereafter, the residual compounds sedimented at the bottoms of the second reactive extraction column were mixed with water or 10% wt. salt solution with a 50:50 weight ratio and transferred to the third reactive extraction column which is filled with the third solvent. In one exemplary embodiment, the salt solution may be prepared using a salt having 10% wt. solubility in water. Salts may include, but are not limited to, calcium chloride, sodium sulfate, sodium acetate, potassium chloride. In the end, about 90% of ethyl acetate produced through the esterification reaction was extracted in the third column. The residual compounds sedimented at the bottoms of the third reactive extraction column comprising ethyl acetate, unreacted ethanol, acetic acid, acid sulfuric and water may be transferred to a distillation unit; then separated ethyl acetate and unreacted ethanol formed an azeotrope with water and were added to the second column as a co-feed. Solvents of all three columns contained ethyl acetate and small amounts of ethanol which were purified to high-purity ethyl acetate using an atmospheric distillation unit. The separated product after distilling each solvent may comprise greater than about 90%, greater than about 95%, greater than about 96%, greater than about 97%, greater than about 98%, greater than about 99%, or greater than about 99.5% ethyl acetate by weight.

### Example 2: Production of High-Purity Methyl Acetate

In this example, methyl acetate was produced by an exemplary reactive extraction system and process similar to exemplary P&ID 100 as presented in FIG. 1. At first, a first mixture of acetic acid, methanol, and acid sulfuric was prepared in a 1000 cc beaker. The temperature of the first mixture was controlled at about 30 °C. The preparation involved mixing 300 (g) of acetic acid (99.9% purity), 10% excess weight of methanol (99.9% purity) about 176 (g), with 3 (g) of acid sulfuric (98% purity), and stirred until well mixed. Then, three 20-inch height and 4-inch diameter glass column were packed with either ceramic saddles, stainless steel pall rings, or trays. In the next step, the first column was filled with the first extraction solvent. The volume of the solvent was 2330 g. It should be mentioned that the volume of the solvent is proportional to separating a 10% wt. methyl acetate solution. Then, the first mixture was added dropwise to the first column using a dropping funnel over 2 hours. Methyl acetate is dissolved in the first solvent as soon as it is formed through the esterification reaction. Then, unreacted reactants comprising methanol, acetic acid, and acid sulfuric with water as a potential byproduct produced by the esterification reaction were sedimented at the bottoms of the first reactive extraction column. In the next step, the residual compounds sedimented at the bottoms of the first reactive extraction column were transferred to a dropping funnel for dropwise adding to the second reactive extraction column which is filled with the second solvent. The esterification reaction was completed in the second column. However, about 80% of the produced methyl acetate was transferred to the solvent phase. Again, the residual compounds were sedimented at the bottoms of the second column which may be evacuated for the next step. Thereafter, the residual compounds sedimented at the bottoms of the second reactive extraction column were mixed with water or 10% wt. salt solution with a 50:50 weight ratio and transferred to the third reactive extraction column which is filled with the third solvent. In one exemplary embodiment, the salt solution may be prepared using a salt having 10% wt. solubility in water. Salts may include, but are not limited to, calcium chloride, sodium sulfate, sodium acetate, potassium chloride. In the end, about 90% of methyl acetate produced through the esterification reaction was extracted in the third column. The residual compounds sedimented at the bottoms of the third reactive extraction column comprising methyl acetate, unreacted methanol, acetic acid, acid sulfuric and water may be transferred to a distillation unit; then separated methyl acetate and unreacted ethanol formed an azeotrope with water and were added to the second column as a co-feed. Solvents of all three columns contained methyl acetate and small amounts of methanol which were purified to high-purity methyl acetate using an atmospheric distillation unit. The separated product after distilling each solvent may comprise greater than about 90%, greater than about 95%, greater than about 96%, greater than about 97%, greater than about 98%, greater than about 99%, or greater than about 99.5% methyl acetate by weight.

GC analysis of methyl acetate produced through the reactive extraction process explained in Example 2 shows that the purity of methyl acetate is 99.5%.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it may be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

1. A process of producing a C1-C2 alkyl acetate with at least 95% purity, comprising:
producing a first mixture by mixing acetic acid, a C1-C2 aliphatic alcohol, and an acid catalyst in first mixer;
producing a first atomized mixture by atomizing the first mixture using a first atomizer;
pumping a first extraction solvent to a first reactive extraction column, wherein the first extraction solvent is a combination of 5% wt. aromatic compound, and 95% wt. of a mixture of naphthenic and paraffinic compounds, wherein the mixture of the naphthenic compound and the paraffinic compound has a weight ratio (the naphthenic compound: the paraffinic compound) ranging from 20:80 to 80:20, wherein the first extraction solvent has a boiling point between 140 °C and 220 °C, and a density between 0.78 g/cm³ and 0.8 g/cm³;
pumping the atomized first mixture to the first reactive extraction column;
producing a first reaction product comprising a C1-C2 alkyl acetate and water in the first reactive extraction column, wherein the conversion of the C1-C2 aliphatic alcohol in the atomized first mixture to the C1-C2 alkyl acetate is at maximum 70%;
removing an overhead stream from the first reactive extraction column by contacting the first reaction product with the first extraction solvent, wherein the overhead stream comprises a first organic phase comprising 10% wt. C1-C2 alkyl acetate produced in the first reaction product that is dissolved in the first extraction solvent;
transferring the first organic phase to a first distillation unit;
separating the first organic phase into C1-C2 alkyl acetate with at least 95% purity and the first extraction solvent using the first distillation unit, wherein separating the first organic phase into C1-C2 alkyl acetate with at least 95% purity and the first extraction solvent using the first distillation unit comprises regenerating the first extraction solvent;
recycling back the regenerated first extraction solvent to the first reactive extraction column;
transferring a first residual compound in the first reactive extraction column to a second atomizer, wherein the first residual compound comprising unreacted acetic acid, the C1-C2 aliphatic alcohol, the acid catalyst, water, and C1-C2 alkyl acetate produced in the first reaction product are atomized;
pumping a second extraction solvent to a second reactive extraction column, wherein the second extraction solvent is a combination of 20% wt. aromatic compound having boiling points between 180 °C and 190 °C, and 80% wt. of a mixture of naphthenic and paraffinic compounds, wherein the mixture of the naphthenic compound and the paraffinic compound has a weight ratio (the naphthenic compound: the paraffinic compound) ranging from 20:80 to 80:20, wherein the second extraction solvent has a boiling point between 150 °C and 220 °C, and a density between 0.72 g/cm³ and 0.74 g/cm³;
pumping the atomized first residual compounds to the second reactive extraction column;
producing a second reaction product comprising a C1-C2 alkyl acetate and water in the second reactive extraction column, wherein the conversion of the C1-C2 aliphatic alcohol in the atomized first residual compounds to the C1-C2 alkyl acetate is at maximum 90%;
removing an overhead stream from the second reactive extraction column by contacting the second reaction product with the second extraction solvent, wherein the overhead stream comprises a second organic phase comprising 10% wt. C1-C2 alkyl acetate produced in the second reaction product that is dissolved in the second extraction solvent;
transferring the second organic phase to a second distillation unit;
separating the second organic phase into C1-C2 alkyl acetate with at least 95% purity and the second extraction solvent using the second distillation unit, wherein separating the second organic phase into C1-C2 alkyl acetate with at least 95% purity and the second extraction solvent using the second distillation unit comprises regenerating the second extraction solvent;
recycling back the regenerated second extraction solvent to the second reactive extraction column;
transferring a second residual compound in the second reactive extraction column to a second mixer, wherein the second residual compounds comprising unreacted acetic acid, the C1-C2 aliphatic alcohol, the acid catalyst, water, and C1-C2 alkyl acetate produced in the second reaction product;
producing a second mixture by adding a 10% wt. salt solution to the second mixer, wherein the second residual compound and 10% wt. salt solution are mixed;
transferring the second mixture to a third atomizer, wherein the second mixture is atomized;
pumping a third extraction solvent to a third reactive extraction column, wherein the third extraction solvent is a combination of 35% wt. aromatic compound, and 65% wt. of a mixture of naphthenic and paraffinic compounds, wherein the mixture of the naphthenic compound and the paraffinic compound has a weight ratio (the naphthenic compound: the paraffinic compound) ranging from 20:80 to 80:20, wherein the second extraction solvent has a boiling point between 150 °C and 180 °C, and a density between 0.77 g/cm³ and 0.79 g/cm³;
pumping the atomized second mixture to the third reactive extraction column;
contacting the atomized second mixture with the third extraction solvent in the third reactive extraction column, wherein the C1-C2 alkyl acetate remained in the atomized second mixture is extracted by the third extraction solvent;
removing an overhead stream from the third reactive extraction column, wherein the overhead stream comprises a third organic phase comprising 10% wt. C1-C2 alkyl acetate remained in the atomized second mixture that is dissolved in the third extraction solvent;
transferring the third organic phase to a third distillation unit;
separating the third organic phase into C1-C2 alkyl acetate with at least 95% purity and the third extraction solvent using the third distillation unit, wherein separating the third organic phase into C1-C2 alkyl acetate with at least 95% purity and the third extraction solvent using the third distillation unit comprises regenerating the third extraction solvent; and
recycling back the regenerated third extraction solvent to the third reactive extraction column.

2. The process of producing methyl acetate with at least 95% purity of claim 1, wherein the producing a first mixture by comprises mixing acetic acid, methanol, and acid sulfuric.

3. The process of producing ethyl acetate with at least 95% purity of claim 1, wherein the producing a first mixture comprises mixing acetic acid, ethanol, and acid sulfuric.

4. The process of producing a C1-C2 alkyl acetate with at least 95% purity of claim 1, wherein reacting acetic acid with C1-C2 aliphatic alcohol comprises reacting C1-C2 aliphatic alcohol with acetic acid with a molar ratio between 1.1 to 1.3, in the presence of at least 1% wt. acid catalyst based on acid acetic.

5. The process of producing a C1-C2 alkyl acetate with at least 95% purity of claim 1, wherein producing the first mixture by mixing acetic acid, a C1-C2 aliphatic alcohol, and an acid catalyst comprises producing the reaction mixture by mixing acetic acid, a C1-C2 aliphatic alcohol, and an acid catalyst at a temperature between 20 °C and 35 °C.
